(19) United States
(12) Patent Application Publication
(10) Pub. No.: US 2026/0305967 A1
(43) Pub. Date: Oct. 8, 2026

(54) MEASURING INTRACRANIAL PRESSURE USING A CEREBROSPINAL FLUID SHUNT DEVICE

(71) Applicant: CHILDREN'S HOSPITAL LOS ANGELES, Los Angeles, CA (US)

(72) Inventors: Matthew BORZAGE, Los Angeles, CA (US); Peter CHIARELLI, Los Angeles, CA (US); Joseph Hyunjong HA, Los Angeles, CA (US)

(73) Assignee: CHILDREN'S HOSPITAL LOS ANGELES, Los Angeles, CA (US)

(21) Appl. No.: 18/996,734

(22) PCT Filed: Aug. 3, 2023
(86) PCT No.: PCT/US2023/029384

§ 371 Date: Jan. 17, 2025

Related U.S. Application Data
(60) Provisional application No. 63/394,911, filed on Aug. 3, 2022.

(51) Int. Cl. A61B 5/03; A61B 5/00; A61B 5/026; A61B 5/055; A61M 27/00

(52) U.S. Cl. CPC A61B 5/031; A61B 5/0263; A61B 5/055; A61B 5/7271; A61M 27/006; A61B 2560/0223; A61M 2205/04; A61M 2205/3334; A61M 2205/50; A61M 2210/0693

(57) ABSTRACT

Intracranial pressure is determined for a patient that has a cerebrospinal fluid (CSF) shunt. Intracranial pressure information is required to manage the care of patients with CSF shunts. Disclosed is a method by which information about intracranial pressures can be obtained using noninvasive measurements, such as an MRI scan. The method provides advantages of avoiding expensive, invasive surgeries and improves clinical patient outcomes due to the ability to monitor intracranial pressure and shunt function, and appropriately titrate patient-specific valve settings.

### CROSS REFERENCE TO RELATED APPLICATIONS

This claims the benefit of U.S. Application No. 63/394,911, filed Aug. 3, 2022, which application is incorporated herein by reference.

### FIELD

The method relates to the field of measuring intracranial pressure, particularly in patients with cerebrospinal fluid (CSF) shunts. The method uses measurements of CSF flow in shunted patients obtained using noninvasive imaging (e.g., magnetic resonance imaging (MRI)) to noninvasively measure intracranial pressure.

### BACKGROUND

Over 300,000 children are newly diagnosed with hydrocephalus annually worldwide. Treatment of hydrocephalus is aimed at reducing intracranial pressure by fluid diversion (e.g., CSF shunting, ventriculo-peritoneal-atrial-pleural-cholecystic shunt). Improvement of symptoms or neurological deficit can be directly related to the improvement of intracranial pressure. Approximately 1 million living Americans have hydrocephalus treated with implanted CSF shunts.

Pediatric hydrocephalus leads to about 40,000 hospitalizations and about 18,000 shunt surgeries annually in the U.S. CSF shunts are a common treatment for hydrocephalus, and up to 40% of implanted shunts malfunction within two years after implantation. An examination of hospital re-admission rates found shunts to be the second most prevalent cause for hospital re-admission within 30 days, with over 84% of shunted children requiring at least one revision surgery over 20 years of follow-up. Standard follow-up for shunted patients at major pediatric institutions includes periodic clinical exam and surveillance imaging (e.g., 1-4 minute rapid screening MRI) to inspect ventricular size, as well as a discussion of clinical symptoms and warning signs for shunt failure. Methods that directly evaluate intracranial pressure or predict impending shunt failure would have a broad clinical interest and direct clinical relevance to patient care, as symptoms of hydrocephalus can overlap with those of other medical conditions evaluated in general practice.

CSF shunts can fail with complete or partial blockage of CSF flow at any time after implantation. When shunt malfunction occurs, CSF does not drain at an adequate rate, and intracranial pressure becomes elevated again. These patients undergo screening using common clinically accepted and/or indirect methods for assessing shunt function or elevated intracranial pressure.

The baseline intracranial pressure (ICP) provided by a functioning CSF shunt can vary from patient to patient. Common methods for assessing elevated ICP are (1) palpating the fontanelle of the skull (only possible in young patients with an open fontanelle), (2) performing a spinal tap to estimate intracranial pressure (invasive; also not possible in patients with obstructive origin of hydrocephalus), (3) performing a procedure for placement of intracranial pressure monitoring probe or intracranial catheter (invasive, and rarely performed), (4) observing indirect signs of increased or decreased pressure such as neurological changes, ophthalmologic exam and radiological findings, each of which can be misleading or highly variable between patients, or (5) performing a percutaneous shunt tap using a thin needle into the shunt valve (which often misestimates pressure or CSF flow in the case of sluggish flow, partial obstruction, or collapsed cerebral ventricles). The gold standard (and the variable that each of these methods seeks to indirectly assess) would be direct knowledge of ICP.

It is desirable to obtain an easier and more effective method of measuring intracranial pressure.

### SUMMARY

A method is provided for measuring intracranial pressure. The method can further be used to detect shunt failure and can be used to choose valve settings to achieve optimal shunt flow for individual patients. One general aspect includes a method for measuring intracranial pressure in a patient having a cerebrospinal fluid (CSF) shunt implant, comprising: setting the CSF shunt to a first flow setting; measuring, at the first flow setting, a first rate of CSF flow; setting the CSF shunt to a second flow setting; measuring, at the second flow setting, a second rate of CSF flow; and calculating the intracranial pressure based on the measured flows.

### BRIEF DESCRIPTION OF THE DRAWINGS

FIG. 1 illustrates an environment in which fluid flow is measured through a shunt in an ex vivo system.

FIG. 2 illustrates an environment in which intracranial pressure is measured in a patient, in vivo, having a cerebrospinal fluid (CSF) shunt.

FIG. 3 is a data flow diagram of a method for measuring fluid flow through a shunt.

FIG. 4 shows a relationship between pressure, shunt values, and flow.

FIG. 5 is a flowchart according to one embodiment for measuring shunt values associated with different settings of the CSF shunt.

FIG. 6 is a flowchart according to another embodiment for calculating intracranial pressure.

FIG. 7 is a flowchart according to still another embodiment for measuring a fixed shunt value associated with the CSF shunt.

FIG. 8 is a flowchart according to another embodiment for determining shunt failure.

FIG. 9 is another embodiment for calibrating intracranial pressure measurements ex vivo.

FIG. 10 is another embodiment for measuring intracranial pressure using the calibration of FIG. 9

FIG. 11 is a computer environment that can be used to implement the embodiments herein.

### DETAILED DESCRIPTION

The disclosed method allows determination of the intracranial pressure of a patient who has a cerebrospinal fluid (CSF) shunt. Intracranial pressure information is required to manage the care of patients with CSF shunts. Disclosed is a method by which information about intracranial pressures can be obtained using noninvasive measurements, such as an MRI scan. The method provides advantages of avoiding expensive, invasive surgeries and improves clinical patient outcomes due to the ability to monitor intracranial pressure and shunt function, and appropriately titrate patient-specific valve settings.

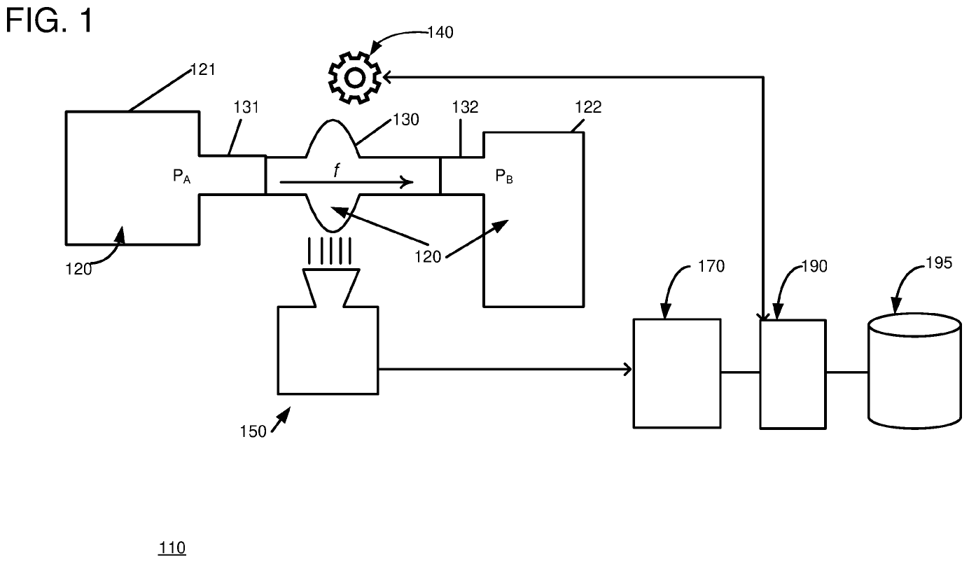

FIG. 1 describes shunt calibration system 110, shown ex vivo, according to one implementation. The shunt calibration system 110 can have one or more chambers 121, 122 at known pressures P_{A}, P_{B}, and conduits 131, 132 (e.g., a catheter) coupling the chambers 121, 122 together through a shunt 130. Cerebrospinal fluid 120 can pass from the chamber 121 at a first pressure, through the first conduit 131, shunt 130 and second conduit 132 to the chamber 122, which is at a lower pressure. Further provided is a shunt valve adjustment tool 140 for adjusting a setting of flow (f) through the shunt. For purposes of explanation, as described herein, modifying a flow setting of the shunt to further restrict the flow of fluid therethrough is described as increasing resistance of the shunt. Conversely, opening the shunt further is described as lowering the resistance. Thus, a changing of resistance is understood to mean modifying a diameter through which fluid flows. For example, the shunt 130 can have a modifiable shunt resistance (\hat{R}) that can be altered using the shunt valve adjustment tool 140. The method for adjusting the setting of \hat{R} can vary depending on the shunt valve type and shunt configuration. A typical shunt that can be used is a variable pressure valve that has multiple adjustable settings that are changeable through using a proximate magnetic field to change an amount of flow through the shunt. For example, an external magnet can be used to rotate a movable piece within the variable pressure valve to change resistance in the valve. With the shunt at a particular setting, based upon the flow through the shunt, a shunt value (analogous to resistance) can be calculated.

A device 150 can measure flow (f) through the shunt 130 in the imaged shunt calibration system 110. Thus, device 150 can obtain information about fluid flow through the CSF shunt using noninvasive imaging. Examples of noninvasive imaging techniques include: magnetic resonance imaging (MRI); phase contrast MRI (PC MRI); x-ray image with injected contrast agent, also commonly called fluoroscopy; computed tomography images with injected contrast agent; ultrasound images with doppler methods to image flow; near infrared spectroscopy imaging; visible light optical methods; thermodilution tracer methods; positron emission tomography; and single photon emission tomography. An MRI scan can be accomplished for example using an MRI scanner manufactured by Phillips, Siemens, GE, Toshiba, Canon, Bruker, Hyperfine, as well as other companies.

The settings on the device 150 can vary according to the manufacturer. The parameters can include patient weight, Field of View (FOV), an acquisition voxel size, a reconstruction voxel size, etc. The device 150 for measuring the flow through the shunt catheter can also include other flow-sensitive MRI sequences, such as arterial spin labelling. Device 150 creates measurements of flow (f) through the shunt 130 at one or more flow settings before it is in a patient. In the case where device 150 is a PC MRI device, a phase contrast image plane parameter is set to be placed orthogonally to a catheter through which CSF is flowing. Additionally, the PC MRI sets one or more phase contrast flow direction parameters to be aligned with a direction of the catheter. The PC MRI can also set a second phase contrast flow direction different from the first phase contrast flow direction parameter. In one example, the PC MRI sets a phase contrast velocity parameter to about 1 centimeter per second or less. The first flow rate and the second flow rate can be measured using a variety of techniques, such as arterial spin labeling magnetic resonance imaging (ASL MRI) other magnetic resonance imaging, ultrasound or other methods for quantifying flow rate.

A module 170 is an aggregator of data flowing from the shunt valve adjustment tool 140 and images captured from device 150. A module 190 is coupled to the aggregator module 170 and can compute the pressure difference between the chambers 121, 122 and the fixed and variable resistance of the shunt 130. The output of the module 190 is stored in a storage device 195 for storing and comparing pressure differences as measured by the system. By connecting the shunt calibration system 110 with one or more chambers 121 and 122 at known pressures P_{A} and P_{B} to a shunt 130 for moving cerebrospinal fluid, a system is created in which fluid flows through the shunt and can be imaged at multiple shunt settings as controlled by shunt valve adjustment tool 140.

The intracranial pressure, flow, and resistance to flow through a CSF shunt system can be defined by the equation:

$${P}_{A j} - {P}_{B j} = [ {f}_{i j} ] \times [ \text{?} + {r}_{j} ] \qquad ( Equation 1 )$$

where P_{A} and P_{B} are pressures at of the chambers 121, 122 at ends of the catheters, fᵢ is the rate of flow through the catheter, \hat{R}ᵢ is the shunt resistance that can be modified in accordance with settings of the shunt according to the shunt valve adjustment tool 140, and r is the shunt and or catheter resistance that cannot be modified (fixed resistance). Variable lowercase i denotes that the resistance can be varied by adjusting the shunt, and variable lowercase j denotes that different test conditions may change the values of the variables.

Equation 1 can be used in many ways, depending on what variables can be adjusted, measured, simplified, or assumed. One simplification of Equation 1 is to represent the difference in pressures P_{A} and P_{B} with a new variable V which is defined by Equation 2.

$$V = {P}_{A} - {P}_{B} \qquad ( Equation 2 )$$

Another simplification of Equation 1 is to assume pressure P_{B} is a pressure that is a useful value. One useful value for pressure P_{B} is the ambient atmospheric pressure, another useful value for P_{B} is the internal pressure in the abdomen or other part of the body. In the case that the chamber at one end of the catheter VA is disposed in a cerebral ventricle and P_{B} is the ambient atmospheric pressure, then P_{A} is the intracranial pressure.

Another useful form of Equation 1 is to assume that the shunt resistance that can be modified \hat{R}ᵢ has more than one setting i and as many as n settings. In this case, a system of equations can be expressed as follows:

$${P}_{A j} - {P}_{B j} = [ {f}_{1 j} ] \times [ {\hat{R}}_{1} + {r}_{j} ]$$

$${P}_{A j} - {P}_{B j} = [ {f}_{2 j} ] \times [ {\hat{R}}_{2} + {r}_{j} ]$$

$${P}_{A j} - {P}_{B j} = [ {f}_{n j} ] \times [ {\hat{R}}_{n} + {r}_{j} ]$$

One approach to solve the system of equations is to assume some of the variables repeated in each equation, are known, or measurable, and that only one unknown variable is in each equation. For example, if P_{Aj}, P_{Bj}, and rⱼ are known, and flow, fᵢⱼ is measurable, then only \hat{R}ᵢ is unknown. Therefore, \hat{R}ᵢ can be computed. One case where this approach is useful is if the shunt has not yet been implanted in a patient and the resistance or resistances of \hat{R}ᵢ can be calculated. A shunt calibration system 110, as illustrated in FIG. 1, can have one or more chambers whose pressures P_{A}, P_{B} can be manipulated and are connected together through catheters and CSF shunt similar to those implanted in patients. In such a system, this method can be used to calculate the variable resistance at different flow settings of the shunt. Additionally, if P_{B} is set to a useful reference value, the value of r is already known, and the value of f can be measured, then the pressure P_{A} can be calculated. One case where this approach would be useful is if the shunt has been implanted in a patient and the patient's intracranial pressure can be calculated. This case can be limited because it assumes that the resistance r has not changed since the shunt was implanted. In any case, the resistance r and the resistances \hat{R}ᵢ can be stored in storage 195.

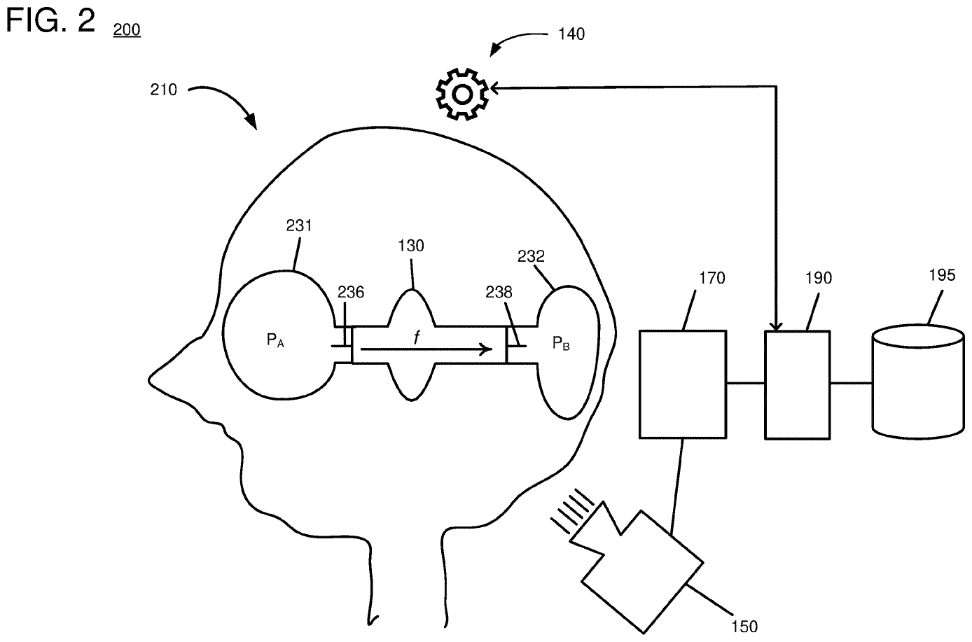

FIG. 2 describes an imaged shunt system 200 in a patient 210 with a shunt 130 inside the patient according to one implementation. The patient 210 can have one or more fluid chambers 231, 232 at unknown pressures P_{A} and P_{B} connected by a shunt 230. Alternatively, P_{B} can be placed at a known pressure, such as atmospheric pressure. Catheters 236, 238 can extend from the shunt 230 into each fluid chamber. For example, catheter 236 is shown extending into fluid chamber 231 and catheter 238 extends into chamber 232. The imaged shunt system can include the tool 140 for adjusting the setting for flow through the shunt 230. For example, the tool 140 can be a magnet that physically turns a movable piece within the shunt 130 to restrict flow (i.e., increase resistance) or to increase flow (i.e., lower resistance).

If P_{B} is a useful reference value (e.g., atmospheric), the value of r is already known, and the value of f can be measured, but the value of intracranial pressure P_{A} and non-modifiable shunt resistance r are unknown. If equation 1 is set to at least i=1 and i=2, where there two known values of \hat{R}ᵢ, and the flows f₁ and f₂ are measured, then this system of equations simplifies as follows:

First, we simplify System of Equations 1 to the case where n=2:

$${P}_{A j} - {P}_{B j} = [ {f}_{1 j} ] \times [ {\hat{R}}_{1} + {r}_{j} ] \qquad ( System of Equations 2 )$$

$${P}_{A j} - {P}_{B j} = [ {f}_{2 j} ] \times [ {\hat{R}}_{2} + {r}_{j} ]$$

Next, the System of Equations 2 is used assuming that Pp is a useful reference value to obtain a System of Equations 3 as follows:

$$V = [ {f}_{1} ] \times [ {\hat{R}}_{1} + r ] \qquad ( System of Equations 3 )$$

$$V = [ {f}_{2} ] \times [ {\hat{R}}_{2} + r ]$$

Next, the System of Equations 3 is expanded using algebra to obtain the System of Equations 4:

$$V = {f}_{1} \times {\hat{R}}_{1} + r \times [ {f}_{1} ] \qquad ( System of Equations 4 )$$

$$V = {f}_{2} \times {\hat{R}}_{2} + r \times [ {f}_{2} ]$$

Next, the equations in System of Equations 4 are combined:

$$2 V = {f}_{1} \times {\hat{R}}_{1} + {f}_{2} \times {\hat{R}}_{2} + r \times [ {f}_{1} + {f}_{2} ]$$

and the unknown variable V is solved for using algebra to obtain Equation 5:

$$V = \frac{1}{2} \times ( {f}_{1} \times {\hat{R}}_{1} + {f}_{2} \times {\hat{R}}_{2} + r \times [ {f}_{1} + {f}_{2} ] ) \qquad ( Equation 5 )$$

Next, equation 5 is used to expand system of equations 4 and obtain system of equations 5:

$$\frac{1}{2} \times ( {f}_{1} \times {\hat{R}}_{1} + {f}_{2} \times {\hat{R}}_{2} + r \times [ {f}_{1} + {f}_{2} ] ) = {f}_{1} \times {\hat{R}}_{1} + r \times [ {f}_{1} ] \qquad ( System of equations 5 )$$

$$\frac{1}{2} \times ( {f}_{1} \times {\hat{R}}_{1} + {f}_{2} \times {\hat{R}}_{2} + r \times [ {f}_{1} + {f}_{2} ] ) = {f}_{2} \times {\hat{R}}_{2} + r \times [ {f}_{2} ]$$

Inspecting System of Equations 5, there is only one unknown variable, which is r. Thus, in this case, the value of r can be calculated. Once the value of r is calculated, it can be used in System of Equations 3 to calculate the pressure difference V. Thus, a determination can be made if the resistance to flow through the shunt has changed, thereby indicating a potential shunt failure. When pressure P_{B} is assumed to be standard atmospheric pressure, the intracranial pressure P_{A} can be calculated. Both measurements indicate information about the patient's current medical status.

Thus, in FIG. 2, the Systems of Equations 2 through 5 can be implemented in module 190 to evaluate intracranial pressure and shunt failure in the patient 210. Subsequent to measurements made using the shunt calibration system 110 of FIG. 1, measurement then focuses on the shunt 130 having been implanted into the patient 210. With the shunt 130 implanted, the tool 140 can be adjusted through multiple settings to change flow of fluid through the shunt. Imaging 150 can capture images related to the flow. The results are aggregated in module 170. Module 190 can then be used to calculate pressure difference across the shunt using the above equations.

The method can include the proximal catheter 236 that extends from an intracranial fluid space to the CSF flow or pressure regulation valve 130, followed by a second catheter 232 extending from the valve to a fluid absorption space (e.g., abdomen, intravenous system, pleura, gallbladder). Measuring CSF flow rate can be accomplished within the catheters 236, 238 at one or multiple valve resistance settings, which will each yield multiple discrete CSF flow rates. The first rate of CSF flow and additional rates of CSF flow at the different valve resistance settings are measured using PC MRI or one of the other imaging techniques described above. One or more parameters of the PC MRI are tuned to measure CSF flow.

Some shunts allow the adjustment of the amount of flow through them, which changes intracranial pressure. Patients can often have an ideal shunt flow setting that provides an optimal intracranial pressure (ICP) unique to their brain, at which they feel their best. Furthermore, normal ICP ranges are known to change as patients age, rendering substantially different ideal ICP for an infant compared to a young child, teenager, or adult.

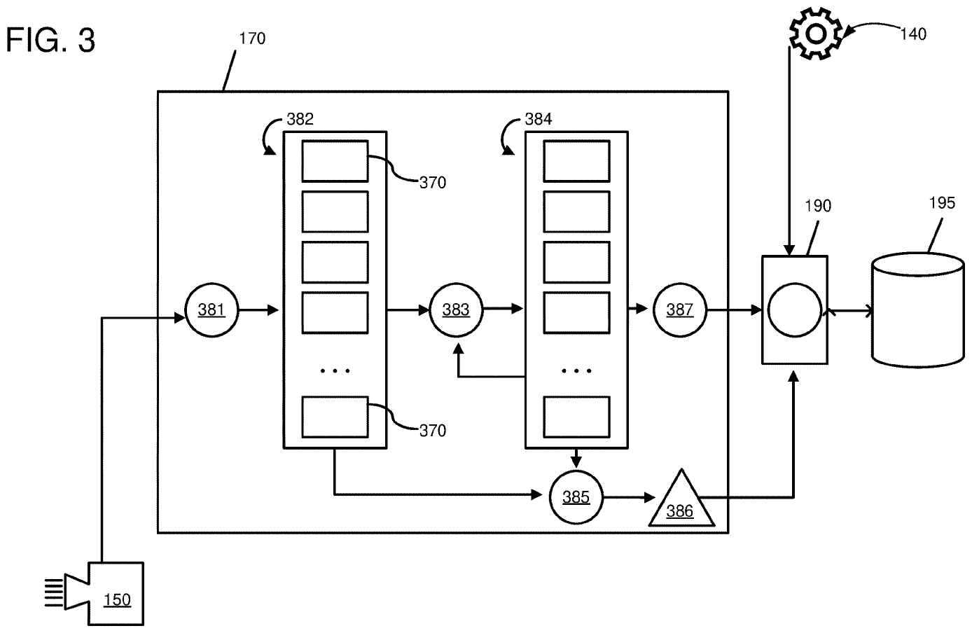

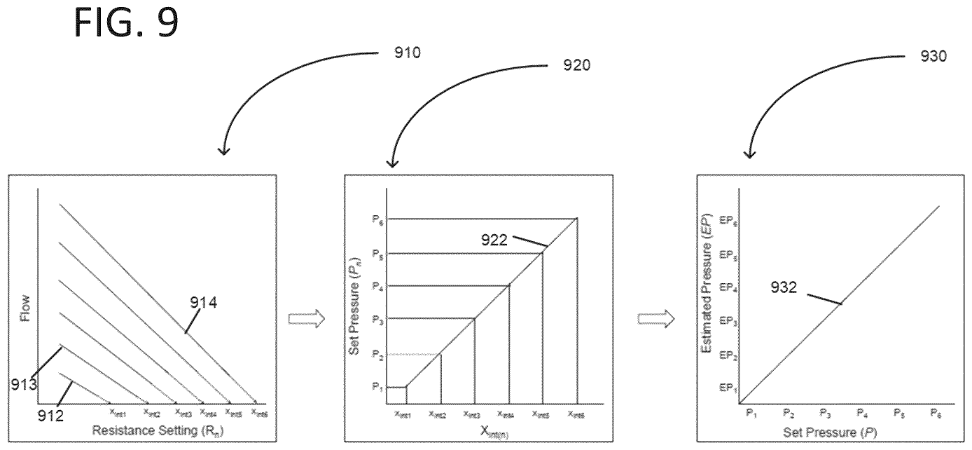

FIG. 3 further illustrates further details of module 170, which can operate within a server computer (such as is shown in FIG. 9). In process 381, imaging data is received from the device 150. The imaging data received at 381 is for a particular setting of flow through the shunt 130, and the setting data is received at 140. A plurality of raw images 382 are generated from the imaging data 381, with individual images shown at 370. The raw images 382 can include magnitude images, phase images, phase difference images, complex difference images, and other types of images. At process 383, processing is performed on the images 382 to generate processed images 384. The processed images 384 can include image labeling indicating where there is and is not motion. The processing for determining motion can include thresholding, edge detection, machine learning, u-nets, and other approaches. One or more of the processed images 384 can further provide indications where voxel intensity is proportional to the velocity of the motion, where the lumen of the shunt is and is not, and other characteristics of the shunt 130 within the patient 210. A flow computation 387 can be computed from the processed images 384, wherein the flow computation can use the voxel intensity data, the state of the lumen, etc. Further processing 385 on the images 384 can be performed to determine physical parameters 386, such as a characterization of the location of the lumen, a diameter of the lumen, an eccentricity of the lumen, a signal-to-noise ratio in the images, a contrast-to-noise ratio in the images, and other physical descriptions. Output from computation 387 of flow and the physical parameters 386 can be output to the module 190 to calculate the pressure difference across the shunt. The method to calculate the pressure difference across the shunt can also receive the current shunt settings 140 for adjusting the setting for flow through the shunt. In some embodiments, the current shunt settings are set by module 190 or the server computer containing module 190. In such a case, the shunt settings do not need to be retrieved remotely, but are already stored locally by module 190. The module 190 can use any of the equations (e.g., the system of equations 3) to calculate an intracranial pressure in chamber 231. For example, the current shunt settings 140 can be used to obtain predetermined resistance values \hat{R}ᵢ, which may be stored in storage 195. Additionally, the second pressure chamber can be set to atmospheric pressure. Accordingly, the remaining unknowns can be computed with at least two different shunt settings 140 and two different flow results 386, 387 associated with the resistance settings.

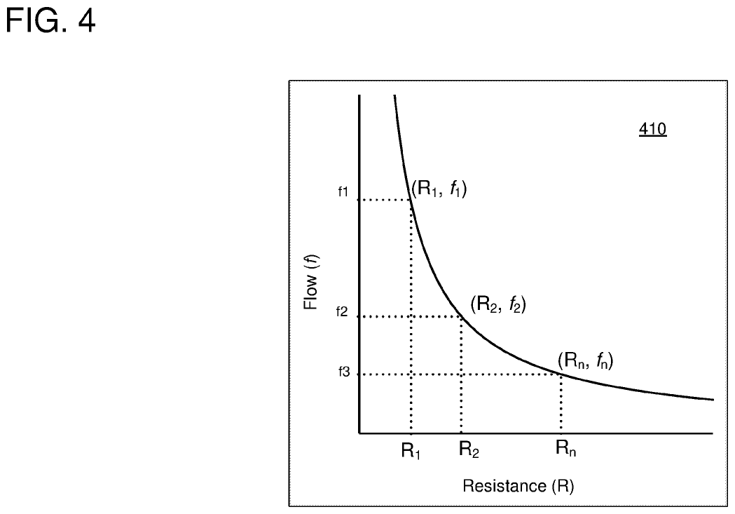

FIG. 4 illustrates the relationship between resistance and flow. A plot 410 shows the relationship between resistance and flow, where the horizontal axis is the resistance through the shunt 130 at three different settings 140. The vertical axis is the flow measured by device 150. The pressures (P_{A} and P_{B}) can be observed under different conditions during calibration, as described with respect to FIG. 1. The resistance values associated with the flows can then be computed and stored in storage 195 to be used in vivo. Additionally, the fitted curve can further used to predict pressures using the equations described above.

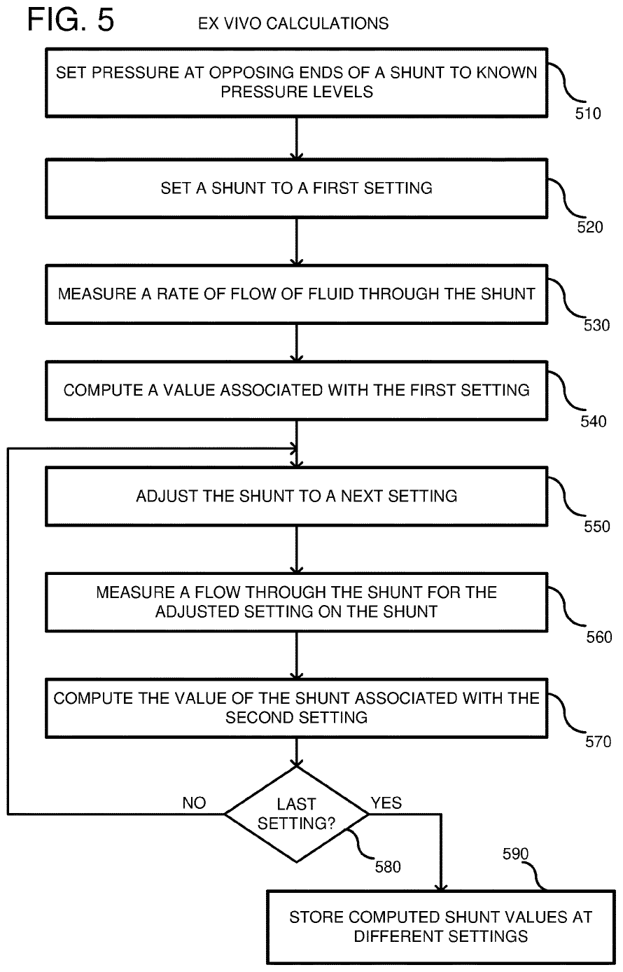

FIG. 5 is a flowchart according to one embodiment for performing ex vivo calculations of shunt values (i.e., resistance) at different settings. In process block 510, a pressure is set at opposing ends of a shunt to known pressure levels. For example, in FIG. 1, a pressure can be set in chamber 121 and in chamber 122. In process block 520, a shunt is set to a first setting. In some cases, the shunt can be fully opened so that the fixed resistance (discussed above) can be computed. As shown in FIG. 1, the module 190 can either control the setting 140 or can display what setting should be manually performed. In process block 530, a rate of flow of fluid through the shunt is measured. For example, in FIG. 1, the detector 150 can be used to image the flow (f) through the shunt 130. In process block 540, the shunt value (e.g., resistance) associated with the setting can be computed using the known pressure and flow. Using the formulas described above, with pressure and flow known, the shunt value can be determined at the associated shunt setting. If the shunt is assumed to be fully opened, then the resistance can be considered the fixed resistance level. At process block 550, the shunt is adjusted to a next setting. For example, the adjustment can be made at 140 (FIG. 1) by using a magnet or other means for changing settings on the shunt 130. Typically, the magnet can be used to rotate a valve within the shunt to further restrict or open the flow. In process block 560, a flow through the shunt is measured at the new setting as previously described. At process block 570, the shunt value associated with the new setting can be computed using the equations previously described. In decision block 580, a determination is made whether the desired settings of the shunt have been analyzed. If not, then process blocks 550, 560 and 570 are repeated with a new shunt setting. If decision block 580 is answered in the affirmative, then the computed shunt settings are stored in association with the computed shunt values (process block 590).

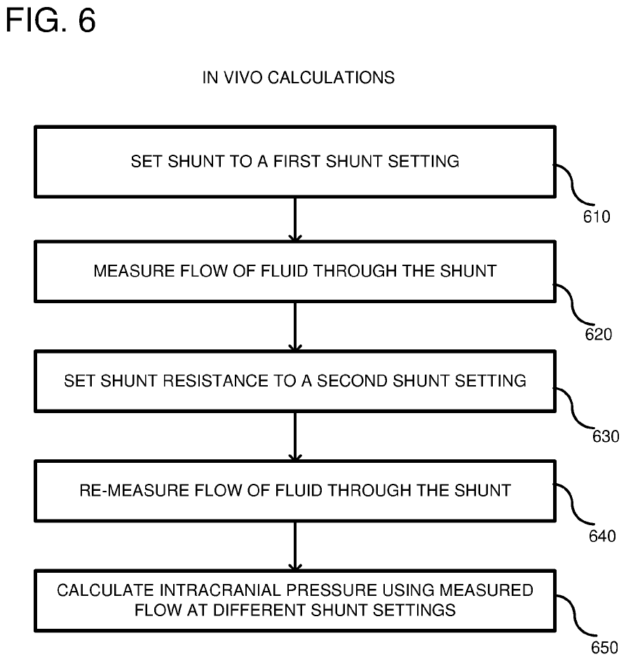

FIG. 6 is a flowchart illustrating in vivo calculations with the shunt inserted into a patient. In process block 610, a shunt is set to a first setting. For example, at 140 in FIG. 2, the shunt 130 can be adjusted, such as through using magnets. In process block 620, flow is measured through the shunt at the current shunt setting. For example, in FIG. 2, the device 150 can image the flow (f) through the shunt 130. In process block 630, the shunt can be adjusted again to a second shunt setting, different than the first setting. At process block 640, the flow of fluid is again measured through the shunt, such as by using device 150. In process block 650, using the fluid flow and the different values associated with the shunt settings, an intracranial pressure can be calculated. For example, using the equations described above, two equations can be calculated with two unknowns. One of the unknowns is the intracranial pressure P_{A} in FIG. 2, which can be computed (P_{B} can be set to a known value, such as atmospheric pressure). Although two shunt settings and two different measured flows are used, additional shunt settings and measurements can be used. For example, it is desirable to have different measured flows and the shunt settings can be changed any number of times to ensure that different measured flows are obtained.

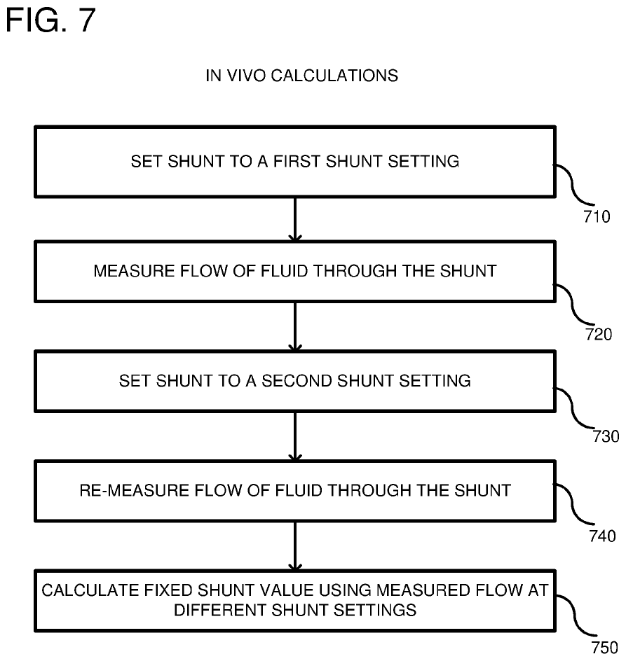

FIG. 7 is a flowchart illustrating in vivo calculations with the shunt inserted into a patient according to another embodiment. In process block 710, shunt is set to a first setting. For example, at 140 in FIG. 2, the shunt 130 can be adjusted, such as through using magnets. In process block 720, flow is measured through the shunt at the current shunt setting. For example, in FIG. 2, the device 150 can image the flow (f) through the shunt 130. In process block 730, the shunt can be adjusted again to a second setting, different than the first setting. At process block 740, the flow of fluid is again measured through the shunt, such as by using device 150. In process block 750, using the fluid flow and the different values associated with the shunt settings, a fixed resistance (r) can be calculated. For example, using the equations described above, two equations can be calculated with two unknowns. One of the unknowns is the intracranial pressure P_{A} in FIG. 2, which can be computed (P_{B} can be set to a known value, such as atmospheric pressure). The only remaining unknown (once pressure is determined) is the fixed resistance (r), which can be computed. The fixed resistance represents the shunt value with the shunt fully opened.

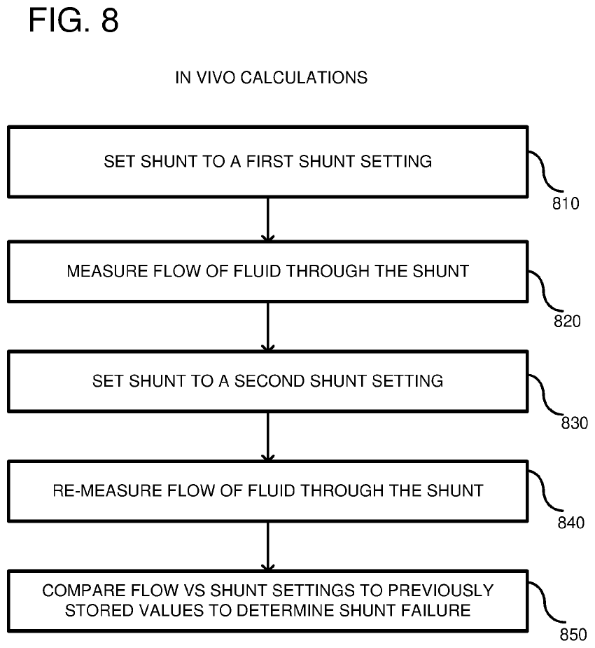

FIG. 8 is a flowchart illustrating in vivo calculations with the shunt inserted into a patient according to another embodiment. In process block 810, shunt is set to a first setting. For example, at 140 in FIG. 2, the shunt 130 can be adjusted, such as through using magnets. In process block 820, flow is measured through the shunt at the current shunt setting. For example, in FIG. 2, the device 150 can image the flow (f) through the shunt 130. In process block 830, the shunt can be adjusted again to a second setting, different than the first setting (a different diameter setting). At process block 840, the flow of fluid is again measured through the shunt, such as by using device 150. In process block 850, the flow and shunt values can be used to determine if shunt failure is occurring. For example, the flow and shunt values can be compared to previous values stored in database 195. The curve of FIG. 4 represents an expected flow vs resistance level and if the measured values are not inline with these previously recorded values, then the shunt can be faulty. In particular, if the measured values exceed the expected values by more than a threshold amount, an alert can be made that the shunt is faulty.

FIG. 9 is another embodiment for calibrating for measuring ICP, ex vivo. First, at 910, a first pressure is used, such as in chamber 121 (FIG. 1) and the setting of the shunt is changed through multiple settings using adjustment tool 140. A flow is measured at each setting using measurement device 150. The results can be plotted as illustrated by line 912 and the line slope can be used to determine an X intercept (Xᵢₙₜ₁). The pressure in chamber 121 can be changed to a second pressure, greater than the first pressure, and the shunt is changed again through multiple settings with flow measured at each setting to obtain a line 913. A second X intercept (Xᵢₙₜ₂) can then be determined using an equation for a line 913. This process can be repeated for any desired number of pressure settings. FIG. 9 shows six different pressure settings are used to obtain six X intercepts, the final one of which is (Xᵢₙₜ₆), which is obtained using an equation of line 914.

A second graph 920 is generated using the calculate six X intercepts and the associated pressure settings for each X intercept. Each point is plotted and the resultant plot 922 can be a curve or line and is called, generically, the calibration curve. The equation of the calibration curve 922 serves as a calibration for estimating pressure against valves of the same make and model used in vivo. A third plot 930 uses the pressure settings (from plot 920) along the X-axis to determine estimate pressures. The resultant line 932 can have a slope of 1 and can be used to determine whether shunts are failing when compared to a similar in vivo plot. All of the calculations for FIG. 9 can be performed in module 190 of FIG. 1.

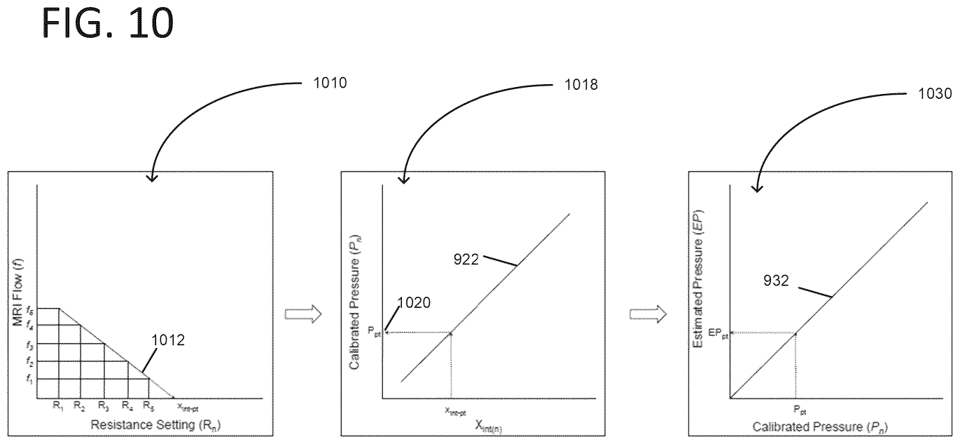

FIG. 10 uses the calibration performed ex vivo to determine ICP in vivo. At 1010, a shunt, of the same make and model as was used to obtain the data in FIG. 9, is used to obtain flow through the shunt are various settings of the shunt in the patient. Thus, the shunt is placed in a setting corresponding to a resistance value of R₁ and a flow f₁ is taken. This process is repeated for multiple settings (at least 2). An equation of the resulting line 1012 can be used to determine the intersection with the X axis as is shown at point Xᵢₙₜ₋ₚₜ. The calibration curve 922 (from FIG. 9) is used in graph 1018 with the Xᵢₙₜ₋ₚₜ plotted along the X axis to determine a corresponding ICP within the patient, as shown at 1020. Thus, at least two different flows can be measured at different shunt settings, and, from this, the intracranial pressure of a patient can be determined. The graph 1030 includes the calibrated line 932. The resultant ICP 1020 can be plotted as an X coordinate against the line 932 to find an estimated pressure in vivo. The estimated pressure can be used to determine if the shunt is degrading. The calculations can likewise be performed by the module 190 of FIG. 1.

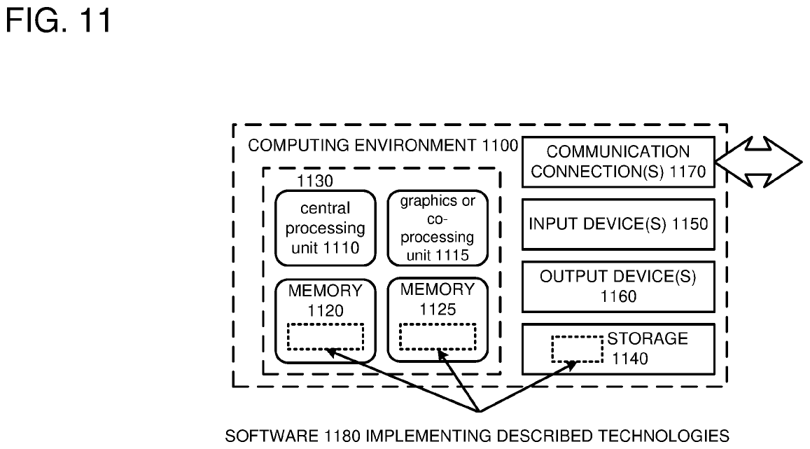

FIG. 11 depicts a generalized example of a suitable computing environment 1100 in which the described innovations may be implemented. The computing environment 1100 is not intended to suggest any limitation as to scope of use or functionality, as the innovations may be implemented in diverse general-purpose or special-purpose computing systems. For example, the computing environment 1100 can be any of a variety of computing devices (e.g., desktop computer, laptop computer, server computer, tablet computer, etc.)

With reference to FIG. 11, the computing environment 1100 includes one or more processing units 1110, 1115 and memory 1120, 1125. In FIG. 11, this basic configuration 1130 is included within a dashed line. The processing units 1110, 1115 execute computer-executable instructions. A processing unit can be a general-purpose central processing unit (CPU), processor in an application-specific integrated circuit (ASIC) or any other type of processor. In a multi-processing system, multiple processing units execute computer-executable instructions to increase processing power. For example, FIG. 11 shows a central processing unit 1110 as well as a graphics processing unit or co-processing unit 1115. The tangible memory 1120, 1125 may be volatile memory (e.g., registers, cache, RAM), non-volatile memory (e.g., ROM, EEPROM, flash memory, etc.), or some combination of the two, accessible by the processing unit(s). The memory 1120, 1125 stores software 1180 implementing one or more innovations described herein, in the form of computer-executable instructions suitable for execution by the processing unit(s). For example, the memory can store modules 170, 190 for image processing and computing intracranial pressure as described above.

A computing system may have additional features. For example, the computing environment 1100 includes storage 1140, one or more input devices 1150, one or more output devices 1160, and one or more communication connections 1170. An interconnection mechanism (not shown) such as a bus, controller, or network interconnects the components of the computing environment 1100. Typically, operating system software (not shown) provides an operating environment for other software executing in the computing environment 1100, and coordinates activities of the components of the computing environment 1100.

The tangible storage 1140 may be removable or non-removable, and includes magnetic disks, magnetic tapes or cassettes, CD-ROMs, DVDs, or any other medium which can be used to store information in a non-transitory way and which can be accessed within the computing environment 1100. The storage 1140 stores instructions for the software 1180 implementing one or more innovations described herein.

The input device(s) 1150 may be a touch input device such as a keyboard, mouse, pen, or trackball, a voice input device, a scanning device, or another device that provides input to the computing environment 1100. The output device(s) 1160 may be a display, printer, speaker, CD-writer, or another device that provides output from the computing environment 1100.

The communication connection(s) 1170 enable communication over a communication medium to another computing entity. The communication medium conveys information such as computer-executable instructions, audio or video input or output, or other data in a modulated data signal. A modulated data signal is a signal that has one or more of its characteristics set or changed in such a manner as to encode information in the signal. By way of example, and not limitation, communication media can use an electrical, optical, RF, or other carrier.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

Any of the disclosed methods can be implemented as computer-executable instructions stored on one or more computer-readable storage media (e.g., one or more optical media discs, volatile memory components (such as DRAM or SRAM), or non-volatile memory components (such as flash memory or hard drives)) and executed on a computer (e.g., any commercially available computer, including smart phones or other mobile devices that include computing hardware). The term computer-readable storage media does not include communication connections, such as signals and carrier waves. Any of the computer-executable instructions for implementing the disclosed techniques as well as any data created and used during implementation of the disclosed embodiments can be stored on one or more computer-readable storage media. The computer-executable instructions can be part of, for example, a dedicated software application or a software application that is accessed or downloaded via a web browser or other software application (such as a remote computing application). Such software can be executed, for example, on a single local computer (e.g., any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, aspects of the disclosed technology can be implemented by software written in C++, Java, Perl, or any other suitable programming language. Likewise, the disclosed technology is not limited to any particular computer or type of hardware. Certain details of suitable computers and hardware are well known and need not be set forth in detail in this disclosure.

It should also be well understood that any functionality described herein can be performed, at least in part, by one or more hardware logic components, instead of software. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), etc.

Furthermore, any of the software-based embodiments (comprising, for example, computer-executable instructions for causing a computer to perform any of the disclosed methods) can be uploaded, downloaded, or remotely accessed through a suitable communication means. Such suitable communication means include, for example, the Internet, the World Wide Web, an intranet, software applications, cable (including fiber optic cable), magnetic communications, electromagnetic communications (including RF, microwave, and infrared communications), electronic communications, or other such communication means.

The disclosed methods, apparatus, and systems should not be construed as limiting in any way. Instead, the present disclosure is directed toward all novel and nonobvious features and aspects of the various disclosed embodiments, alone and in various combinations and subcombinations with one another. The disclosed methods, apparatus, and systems are not limited to any specific aspect or feature or combination thereof, nor do the disclosed embodiments require that any one or more specific advantages be present or problems be solved.

In view of the many possible embodiments to which the principles of the disclosed invention may be applied, it should be recognized that the illustrated embodiments are only examples of the invention and should not be taken as limiting the scope of the invention. We therefore claim as our invention all that comes within the scope of these claims.

1. A method for measuring intracranial pressure in a patient having a cerebrospinal fluid (CSF) shunt implant, the method comprising:
setting the CSF shunt to a first flow setting;
measuring, at the first flow setting, a first rate of CSF flow through the CSF shunt;
setting the CSF shunt to a second flow setting;
measuring, at the second flow setting, a second rate of CSF flow through the CSF shunt; and
calculating the intracranial pressure using the measured first rate of CSF flow and the second rate of CSF flow.

2. The method of claim 1, wherein the first flow rate and the second flow rate are measured using a phase contrast magnetic resonance imaging (PC MRI) device.

3. The method of claim 2, wherein the PC MRI device has one or more phase contrast image plane parameters to be placed orthogonally to a direction of the first and second flow.

4. The method of claim 2, wherein the PC MRI has one or more phase contrast flow direction parameters aligned with a direction of the first and second flow.

5. The method of claim 1, further including determining a first shunt value and a second shunt value using the CSF shunt, ex vivo, with known pressure values on opposed ends of the CSF shunt and using the first and second shunt values in calculating the intracranial pressure.

6. The method of claim 5, comparing the first and second shunt values and the measured first and second rates of CSF flow to previously measured rates of CSF flow and determining that the CSF shunt is failing based upon the comparison.

7. The method of claim 1, further including determining a calibration curve, ex vivo, using a plurality of measured flows at different flow settings and determining a plurality of resistance settings associated with the plurality of measured flows, wherein the calibration curve is based on the plurality of resistance settings and pressures associated therewith.

8. The method of claim 7, wherein the calculating of the intracranial pressure includes using the first rate of CSF flow and the second rate of CSF flow to determine a resistance setting and using the calibration curve to determine the intracranial pressure.

9. The method of claim 1, further including determining a fixed resistance value with the CSF shunt in a flow setting having a maximum flow rate.

10. The method of claim 1, wherein the first flow setting and the second flow setting each control an amount of flow to pass through the CSF shunt.

11. A method, comprising:
measuring a first flow rate across a cerebrospinal fluid (CSF) shunt at a first flow setting of the CSF shunt;
measuring a second flow rate at a second flow setting of the CSF shunt; and
measuring an intracranial pressure of a patient using at least the first and second flow rates.

12. The method of claim 11, wherein the first rate of CSF flow and the second rate of CSF flow are measured using phase contrast magnetic resonance imaging (PC MRI).

13. The method of claim 11, further including retrieving predetermined CSF shunt values associated with the first and second flow settings and calculating the intracranial pressure using the CSF shunt values and the measured first and second flow rates.

14. The method of claim 11, further including generating a calibration curve, ex vivo, and using the calibration curve for the measuring of the intracranial pressure.

15. The method of claim 11, wherein the calibration curve is generated, ex vivo, using a known pressure and modifying the settings of the shunt using the known pressure to measure resistance.

16. A system, comprising:
a device for measuring flow through a shunt;
a shunt adjustment tool for changing flow through the shunt; and
a server computer for measuring intracranial pressure of a patient using at least two different settings of the shunt using the shunt adjustment tool and at least two different flow measurements.

17. The system of claim 16, wherein the server computer is configured to measure the intracranial pressure by using a calibration curve, which is determined ex vivo, and is configured to calculate a resistance value to plot on the calibration curve.

18. The system of claim 16, wherein the shunt has multiple settings to change a flow of fluid therethrough.

19. The system of claim 16, wherein the shunt adjustment tool includes a magnet for changing a setting on the shunt.

20. The system of claim 16, wherein the device for measuring flow includes a phase contrast magnetic resonance imaging (PC MRI) device.
